(19) United States
(12) Patent Application Publication
(10) Pub. No.: US 2005/0245532 A1
(43) Pub. Date: Nov. 3, 2005

(54) Inhibitors of the 11-beta-hydroxysteroid dehydrogenase Type 1 enzyme and their therapeutic application

(76) Inventors: Ethan D. Hoff, Racine, WI (US); James T. Link, Evanston, IL (US); Marina A. Pliushchev, Vernon Hills, IL (US); Jeffrey J. Rohde, Evanston, IL (US); Martin Winn, Deerfield, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/835,132

(22) Filed: Apr. 29, 2004

(51) Int. Cl. A61K 31/495; A61K 31/496; A61K 31/445

(52) U.S. Cl. 514/252.12; 514/254.1; 514/319

(57) ABSTRACT

The present invention relates to the use of inhibitors of the 11-beta-hydroxysteroid dehydrogenase Type 1 enzyme. The present invention further relates to the use of inhibitors of 11-beta-hydroxysteroid dehydrogenase Type 1 enzyme for the treatment or prophylactically treatment of non-insulin dependent type 2 diabetes, insulin resistance, obesity, lipid disorders, metabolic syndrome, and other diseases and conditions mediated by excessive glucocorticoid action.

### FIELD OF INVENTION

The present invention relates to the use of inhibitors of the 11-beta-hydroxysteroid dehydrogenase Type 1 enzyme. The present invention further relates to the use of inhibitors of 11-beta-hydroxysteroid dehydrogenase Type I enzyme for the treatment of non-insulin dependent type 2 diabetes, insulin resistance, obesity, lipid disorders, metabolic syndrome, and other diseases and conditions that are mediated by excessive glucocorticoid action.

### BACKGROUND OF THE INVENTION

Insulin is a hormone that modulates glucose and lipid metabolism. Impaired action of insulin (insulin resistance) results in reduced insulin-induced glucose uptake, oxidation and storage, reduced insulin-dependent suppression of fatty acid release from adipose tissue (lipolysis), and reduced insulin-mediated suppression of hepatic glucose production and secretion. Insulin resistance frequently occurs in diseases that lead to increased and premature morbidity and mortality.

Diabetes mellitus is characterized by an elevation of plasma glucose levels (hyperglycemia) in the fasting state or after administration of glucose during a glucose tolerance test. While this disease may be caused by several underlying factors, it is generally grouped into two categories, Type 1 and Type 2 diabetes. Type 1 diabetes (or insulin dependent diabetes mellitus, IDDM) is caused by a reduction of production and secretion of insulin. In type 2 diabetes, also referred to as non-insulin dependent diabetes mellitus, or NIDDM, insulin resistance is a significant pathogenic factor in the development of hyperglycemia. Typically, the insulin levels in type 2 diabetes patients are elevated (i.e., hyperinsulinemia), but this compensatory increase is not sufficient to overcome the insulin resistance. Persistent or uncontrolled hyperglycemia in both type 1 and type 2 diabetes mellitus is associated with increased incidence of macrovascular and/or microvascular complications including atherosclerosis, coronary heart disease, peripheral vascular disease, stroke, nephropathy, neuropathy, and retinopathy.

Insulin resistance, even in the absence of profound hyperglycemia, is a component of the metabolic syndrome. Recently, diagnostic criteria for metabolic syndrome have been established. To qualify a patient as having metabolic syndrome, three out of the five following criteria must be met: elevated blood pressure above 130/85 mmHg, fasting blood glucose above 110 mg/dl, abdominal obesity above 40″ (men) or 35″ (women) waist circumference, and blood lipid changes as defined by an increase in triglycerides above 150 mg/dl or decreased HDL cholesterol below 40 mg/dl (men) or 50 mg/dl (women). It is currently estimated that 50 million adults, in the US alone, fulfill these criteria. That population, whether or not they develop overt diabetes mellitus, are at increased risk of developing the macrovascular and microvascular complications of type 2 diabetes listed above.

Available treatments for type 2 diabetes have recognized limitations. Diet and physical exercise can have profound beneficial effects in type 2 diabetes patients, but compliance is poor. Even in patients having good compliance, other forms of therapy may be required to further improve glucose and lipid metabolism.

One therapeutic strategy is to increase insulin levels to overcome insulin resistance. This may be achieved through direct injection of insulin or through stimulation of the endogenous insulin secretion in pancreatic beta cells. Sulfonylureas (e.g., tolbutamide and glipizide) or meglitinide are examples of drugs that stimulate insulin secretion (insulin secretagogues) thereby increasing circulating insulin concentrations high enough to stimulate insulin-resistant tissue. However, insulin and insulin secretagogues may lead to dangerously low glucose concentrations (i.e., hypoglycemia). In addition, insulin secretagogues frequently lose therapeutic potency over time.

Two biguanides, metformin and phenformin, may improve insulin sensitivity and glucose metabolism in diabetic patients. However, the mechanism of action is not well understood. Both compounds may lead to lactic acidosis and gastrointestinal side effects (e.g, nausea or diarrhea).

Alpha-glucosidase inhibitors (e.g., acarbose) may delay carbohydrate absorption from the gut after meals, which may in turn lower blood glucose levels, particularly in the postprandial period. Like biguanides, these compounds may also cause gastrointestinal side effects.

Glitazones (i.e. 5-benzylthiazolidine-2,4-diones) are a newer class of compounds used in the treatment of type 2 diabetes. These agents may reduce insulin resistance in multiple tissues thus lowering blood glucose. The risk of hypoglycemia may also be avoided. Glitazones modify the activity of the peroxisome proliferator activated receptor (PPAR) gamma subtype. PPAR is currently believed to be the primary therapeutic target for the main mechanism of action for the beneficial effects of these compounds. Other modulators of the PPAR family of proteins are currently in development for the treatment of type 2 diabetes and/or dyslipidemia. Marketed glitazones suffer from side effects including bodyweight gain and peripheral edema.

Additional treatments to normalize blood glucose levels in patients with diabetes mellitus are needed. As a result other therapeutic strategies are being explored including: glucagon-like peptide 1 (GLP-1) analogues and inhibitors of dipeptidyl peptidase IV which increase insulin secretion, inhibitors of key enzymes involved in the hepatic glucose production and secretion (e.g., fructose-1,6-bisphosphatase inhibitors), and direct modulation of enzymes involved in insulin signaling (e.g., protein tyrosine phosphatase-1B, PTP-1B).

Another method of treating or prophylactically treating diabetes mellitus is using inhibitors of 11-β-hydroxysteroid dehydrogenase Type 1 (11-HSD1), as outlined in J. R. Seckl et al., Endocrinology, 142: 1371-1376, 2001, and references cited therein. Glucocorticoids are steroid hormones that are potent regulators of glucose and lipid metabolism. Excessive glucocorticoid action may lead to insulin resistance, type 2 diabetes, dyslipidemia, increased abdominal obesity, and hypertension. Glucocorticoids circulate in the blood in an active form (i.e., cortisol in humans) and an inactive form (i.e., cortisone in humans). 11β-HSD1, which is highly expressed in liver and adipose tissue, converts cortisone to cortisol leading to higher local concentration of cortisol. Inhibition of 11β-HSD1 prevents or decreases the tissue specific amplification of glucocorticoid action thus imparting beneficial effects on blood pressure and glucose- and lipid-metabolism.

Thus, inhibiting 11β-HSD1 would benefit patients suffering from non-insulin dependent type 2 diabetes, insulin resistance, obesity, lipid disorders, metabolic syndrome, and other diseases and conditions mediated by excessive glucocorticoid action.

### SUMMARY OF THE INVENTION

One aspect of the present invention is directed toward a method of inhibiting the 11-beta-hydroxysteroid dehydrogenase Type I enzyme in a mammal, comprising administering a therapeutically effective amount of a compound of formula (I), 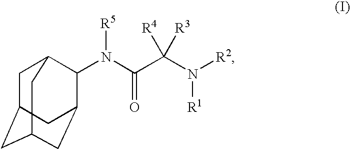
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and the intervening atoms form a cycloalkyl; R³, R⁴ and the intervening atoms form a non-aromatic heterocycle; and, R², R³ and the intervening carbon and nitrogen atoms form a non-aromatic heterocycle; and,
R⁵ is a member selected from the group consisting of hydrogen, alkyl, carboxyalkyl, carboxycycloalkyl, cycloalkyl, aryl, arylalkyl, aryloxyalkyl, heterocycle, heterocyclealkyl, and heterocycleoxyalkyl.

A further aspect of the present invention includes the use of the compounds of formula (I) for the treatment of disorders by inhibiting 11-beta-hydroxysteroid dehydrogenase Type 1 enzyme in a mammal. Such disorders include, but are not limited to, non-insulin dependent type 2 diabetes, insulin resistance, obesity, lipid disorders, metabolic syndrome, and other diseases and conditions mediated by excessive glucocorticoid action.

### DETAILED DESCRIPTION OF THE INVENTION

All patents, patent applications, and literature references cited in the specification are herein incorporated by reference in their entirety.

One particular embodiment of the present invention is directed toward a method of inhibiting the 11-beta-hydroxysteroid dehydrogenase Type I enzyme in a mammal, comprising administering a therapeutically effective amount of a compound of formula (I), 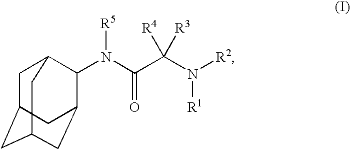
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and the intervening atoms form a cycloalkyl; R³, R⁴ and the intervening atoms form a non-aromatic heterocycle; R², R³ and the intervening carbon and nitrogen atoms form a non-aromatic heterocycle; and,
R⁵ is a member selected from the group consisting of hydrogen, alkyl, carboxyalkyl, carboxycycloalkyl, cycloalkyl, aryl, arylalkyl, aryloxyalkyl, heterocycle, heterocyclealkyl, and heterocycleoxyalkyl.

Another embodiment of the present invention is directed toward a method of inhibiting the 11-beta-hydroxysteroid dehydrogenase Type I enzyme in a mammal, comprising administering a therapeutically effective amount of a compound of formula (II), 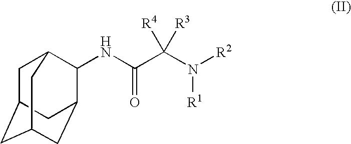
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle; and,
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and the intervening atoms form a cycloalkyl; R³, R⁴ and the intervening atoms form a non-aromatic heterocycle; and, R², R³ and the intervening carbon and nitrogen atoms form a non-aromatic heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IIa), 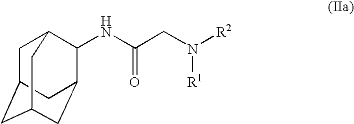
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IIb), 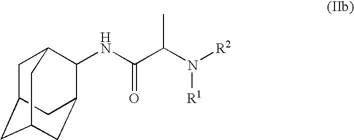
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IIc), 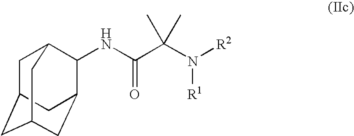
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle.

Another embodiment of the present invention is directed toward a method of inhibiting the 11-beta-hydroxysteroid dehydrogenase Type I enzyme in a mammal, comprising administering a therapeutically effective amount of a compound of formula (III), 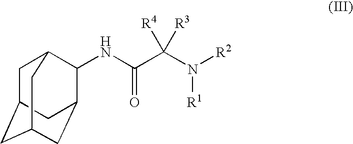
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle; and,
R³ and R⁴ are each a member independently selected from the group consisting of hydrogen, alkyl, carboxyalkyl, carboxycycloalkyl, cycloalkyl, aryl, and, heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IIIa), 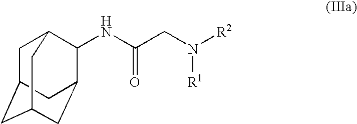
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IIIb), 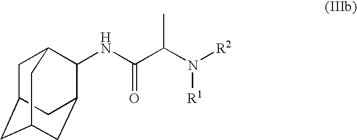
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IIIc), 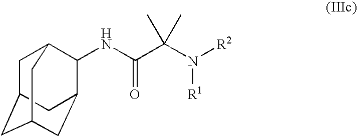
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IV), 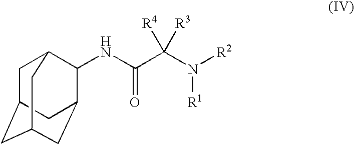
or a therapeutically suitable salt or prodrug thereof, wherein
R¹ and R² taken together with the atom to which they are attached form a heterocycle; and,
R³ and R⁴ are each a member independently selected from the group consisting of hydrogen, alkyl, carboxyalkyl, carboxycycloalkyl, cycloalkyl, aryl, and, heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IVa), 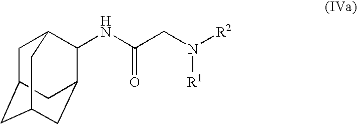
or a therapeutically suitable salt or prodrug thereof, wherein
R¹ and R² taken together with the atom to which they are attached form a heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IVb), 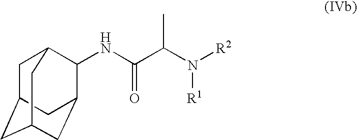
or a therapeutically suitable salt or prodrug thereof, wherein
R¹ and R² taken together with the atom to which they are attached form a heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IVc), 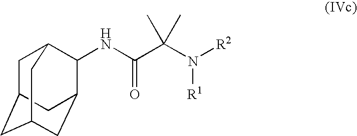
or a therapeutically suitable salt or prodrug thereof, wherein
R¹ and R² taken together with the atom to which they are attached form a heterocycle.

Another embodiment of the present invention is directed toward a method of inhibiting the 11-beta-hydroxysteroid dehydrogenase Type I enzyme in a mammal, comprising administering a therapeutically effective amount of a compound of formula (V), 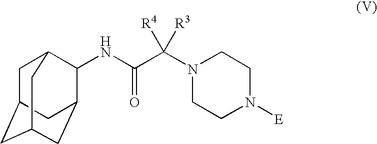
or a therapeutically suitable salt or prodrug thereof, wherein
R³ and R⁴ are each a member independently selected from the group consisting of hydrogen, alkyl, carboxyalkyl, carboxycycloalkyl, cycloalkyl, aryl, heterocycle; R³, R⁴ and the intervening atoms form a cycloalkyl; and R³, R⁴ and the intervening atoms form a non-aromatic heterocycle; and,
E is a member selected from the group consisting of aryl and heterocycle.

Another embodiment of the present invention is directed toward a method of inhibiting the 11-beta-hydroxysteroid dehydrogenase Type I enzyme in a mammal, comprising administering a therapeutically effective amount of a compound of formula (Va), 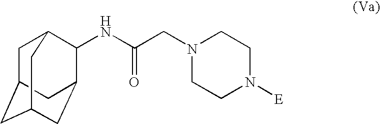
or a therapeutically suitable salt or prodrug thereof, wherein
E is a member selected from the group consisting of aryl and heterocycle.

Another embodiment of the present invention is directed toward a method of inhibiting the 11-beta-hydroxysteroid dehydrogenase Type I enzyme in a mammal, comprising administering a therapeutically effective amount of a compound of formula (Vb), 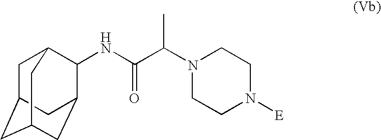
or a therapeutically suitable salt or prodrug thereof, wherein
E is a member selected from the group consisting of aryl and heterocycle.

Another embodiment of the present invention is directed toward a method of inhibiting the 11-beta-hydroxysteroid dehydrogenase Type I enzyme in a mammal, comprising administering a therapeutically effective amount of a compound of formula (Vc), 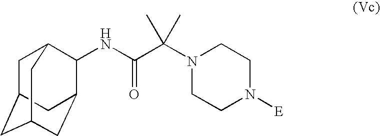
or a therapeutically suitable salt or prodrug thereof, wherein
E is a member selected from the group consisting of aryl and heterocycle.

Another embodiment of the present invention is directed toward a method of inhibiting the 11-beta-hydroxysteroid dehydrogenase Type I enzyme in a mammal, comprising administering a therapeutically effective amount of a compound of formula (Vd), 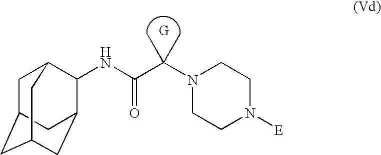
or a therapeutically suitable salt or prodrug thereof, wherein
G is selected from the group consisting of cycloalkyl and non-aromatic heterocycle; and,
E is a member selected from the group consisting of aryl and heterocycle.

Another embodiment of the present invention is directed toward a method of inhibiting the 11-beta-hydroxysteroid dehydrogenase Type I enzyme in a mammal, comprising administering a therapeutically effective amount of a compound of formula (VI), 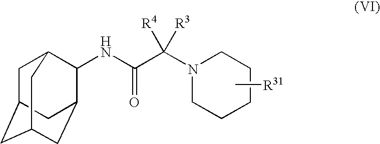
or a therapeutically suitable salt or prodrug thereof, wherein
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and the intervening atoms form a cycloalkyl; R³, R⁴ and the intervening atoms form a non-aromatic heterocycle; and, R², R³ and the intervening carbon and nitrogen atoms form anon-aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIa), 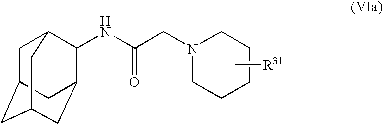
or a therapeutically suitable salt or prodrug thereof, wherein
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIb), 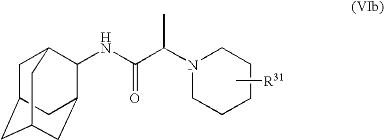
or a therapeutically suitable salt or prodrug thereof, wherein
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIc), 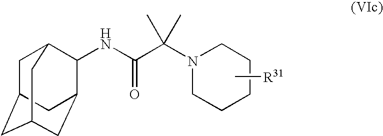
or a therapeutically suitable salt or prodrug thereof, wherein
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VId), 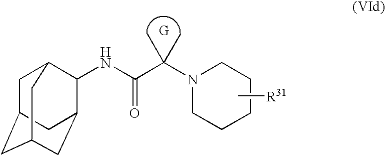
or a therapeutically suitable salt or prodrug thereof, wherein
G is selected from the group consisting of cycloalkyl and non-aromatic heterocycle; and
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VII), 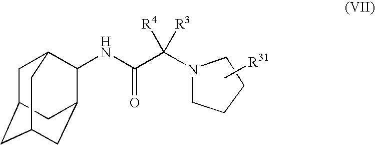
or a therapeutically suitable salt or prodrug thereof, wherein
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and the intervening atoms form a cycloalkyl; and R³, R⁴ and the intervening atoms form a non aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIIa), 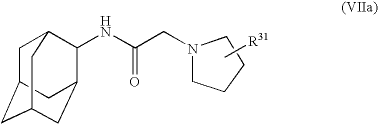
or a therapeutically suitable salt or prodrug thereof, wherein
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIIb), 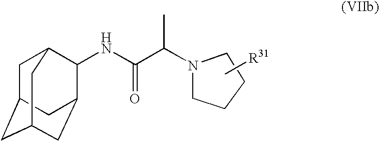
or a therapeutically suitable salt or prodrug thereof, wherein
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIIc), 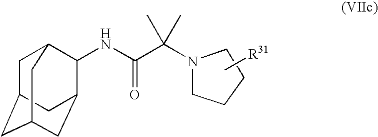
or a therapeutically suitable salt or prodrug thereof, wherein
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIId), 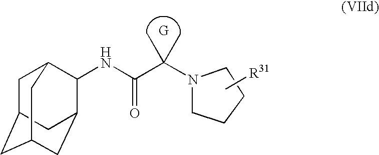
or a therapeutically suitable salt or prodrug thereof, wherein
G is selected from the group consisting of cycloalkyl and non-aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (VIII), 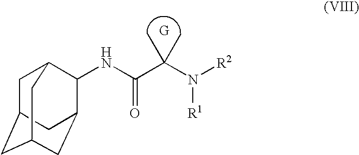
or a therapeutically suitable salt or prodrug thereof, wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle; and,
G is selected from the group consisting of cycloalkyl and non-aromatic heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IX), 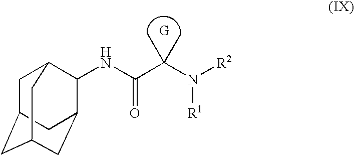
or a therapeutically suitable salt or prodrug thereof, wherein
R¹ and R² taken together with the atom to which they are attached form a heterocycle; and,
G is selected from the group consisting of cycloalkyl and nonaromatic heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IXa), 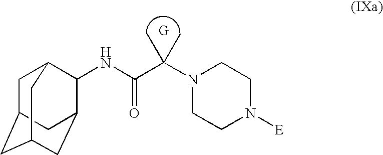
or a therapeutically suitable salt or prodrug thereof, wherein
E is a member selected from the group consisting of aryl and heterocycle; and,
G is selected from the group consisting of cycloalkyl and nonaromatic heterocycle.

Another embodiment of the present invention is directed toward a method of inhibiting the 11-beta-hydroxysteroid dehydrogenase Type I enzyme in a mammal, comprising administering a therapeutically effective amount of a compound of formula (IXb), 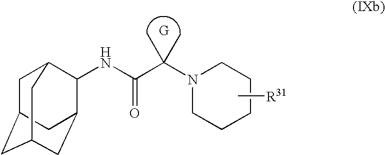
or a therapeutically suitable salt or prodrug thereof, wherein
G is selected from the group consisting of cycloalkyl and non-aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (IXc), 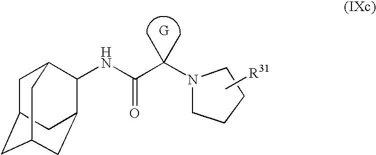
or a therapeutically suitable salt or prodrug thereof, wherein
G is selected from the group consisting of cycloalkyl and non-aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

Another embodiment of the present invention is directed toward a method of inhibiting the 11-beta-hydroxysteroid dehydrogenase Type I enzyme in a mammal, comprising administering a therapeutically effective amount of a compound of formula (X), 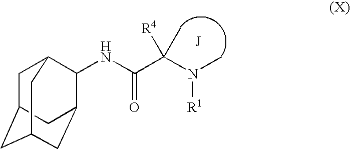
or a therapeutically suitable salt or prodrug thereof, wherein
R¹ is a member selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle;
R⁴ is a member selected from the group consisting of hydrogen, alkyl, carboxyalkyl, carboxycycloalkyl, cycloalkyl, aryl, and, heterocycle; and,
J is a non-aromatic heterocycle.

As set forth herein, the invention includes administering a therapeutically effective amount of any of the compounds of formula I-X and the salts and prodrugs thereof to a mammal. Preferably, the invention also includes administering a therapeutically effective amount of any of the compounds of formula I-X to a human, and more preferably to a human in need of being treated for or prophylactically treated for any of the respective disorders set forth herein.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (I), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (II), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (III), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IV), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (V), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VI), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VII), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VIII), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IX), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (X), wherein the disorder is non-insulin dependent type 2 diabetes.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (I), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (II), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (III), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IV), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (V), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VI), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VII), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VIII), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IX), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (X), wherein the disorder is insulin resistance.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (I), wherein the disorder is obesity

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (II), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (III), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IV), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (V), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VI), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VII), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VIII), wherein the disorder is obesity.

Another aspect of the invention includes method of treating or prophylactically treating disorders, by inhibiting the 11-beta-hydroxysteroid dehydrogenase Type I enzyme in a mammal, comprising administering a therapeutically effective amount of a compound of formula (IX), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (X), wherein the disorder is obesity.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (I), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (II), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (III), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IV), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (V), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VI), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VII), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VIII), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IX), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (X), wherein the disorder is lipid disorders.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (I), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (II), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (III), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IV), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (V), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VI), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VII), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VIII), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IX), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (X), wherein the disorder is metabolic syndrome.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (I), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (II), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (III), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IV), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (V), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VI), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VII), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (VIII), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (IX), wherein the disorders are other diseases and conditions that are mediated by excessive glucocorticoid action.

Another aspect of the invention includes a method of treating or prophylactically treating disorders, by inhibiting the 11-beta-hydroxysteroid dehydrogenase Type I enzyme in a mammal, comprising administering a therapeutically effective amount of a compound of formula (X), wherein the disorders are other diseases and conditions that are mediated by excessive glucocorticoid action.

Definition of Terms

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkyl,” as used herein, refers to a straight or branched chain hydrocarbon containing from 1 to 10 carbon atoms. Representative examples of alkyl include, but are not limited to, methyl, ethyl, r-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkyl-NH,” as used herein, refers to an alkyl group, as defined herein, appended to the parent molecular moiety through a nitrogen atom.

The term “alkyl-NH-alkyl,” as used herein, refers to an alkyl-NH group, as defined herein, appended to the parent molecular moiety through an alkyl group, as defined herein.

The term “aryl,” as used herein, refers to a monocyclic-ring system or a polycyclic-ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention may be optionally substituted with 0, 1, 2, 3, 4 or 5 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, methylenedioxy, nitro, R_{f}R_{g}N—, R_{f}R_{g}Nalkyl, R_{f}R_{g}Ncarbonyl and R_{f}R_{g}Nsulfonyl, wherein R_{f} and R_{g} are members independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aryl-heterocycle,” as used herein, refers to an aryl group, as defined herein, appended to the parent molecular moiety through a heterocycle group, as defined herein.

The term “aryl-NH—,” as used herein, refers to an aryl group, as defined herein, appended to the parent molecular moiety through a nitrogen atom.

The term “aryl-NH-alkyl,” as used herein, refers to an aryl-NH— group, as defined herein, appended to the parent molecular moiety through an alkyl group, as defined herein.

The term “aryloxy,” as used herein, refers to an aryl group, as defined herein, appended to the parent molecular moiety through an oxy moiety, as defined herein. Representative examples of aryloxy include, but are not limited to phenoxy, naphthyloxy, 3-bromophenoxy, 4-chlorophenoxy, 4-methylphenoxy, and 3,5-dimethoxyphenoxy.

The term “aryloxyalkyl,” as used herein, refers to an aryloxy group, as defined herein, appended to the parent molecular moiety through an alkyl group as defined herein.

The term “arylsulfonyl,” as used herein, refers to an aryl group, as defined herein, appended to the parent molecular moiety through a sulfonyl group, as defined herein. Representative examples of arylsulfonyl include, but are not limited to, phenylsulfonyl, 4-bromophenylsulfonyl and naphthylsulfonyl.

The term “carbonyl,” as used herein refers to a —C(O)— group.

The term “carboxy,” as used herein refers to a —C(O)—OH group.

The term “carboxyalkyl,” as used herein refers to a carboxy group as defined herein, appended to the parent molecular moiety through an alkyl group as defined herein.

The term “carboxycycloalkyl,” as used herein refers to a carboxy group as defined herein, appended to the parent molecular moiety through an cycloalkyl group as defined herein.

The term “cycloalkyl,” as used herein, refers to a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycloalkyl groups of this invention may be substituted with 1, 2, 3, 4 or 5 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, nitro, R_{f}R_{g}N—, R_{f}R_{g}Nalkyl, R_{f}R_{g}Ncarbonyl and R_{f}R_{g}Nsulfonyl, wherein R_{f} and R_{g} are members independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “cycloalkylsulfonyl,” as used herein, refers to cycloalkyl group, as defined herein, appended to the parent molecular moiety through a sulfonyl group, as defined herein. Representative examples of cycloalkylsulfonyl include, but are not limited to, cyclohexylsulfonyl and cyclobutylsulfonyl.

The term “halo” or “halogen,” as used herein, refers to —Cl, —Br, —I or —F.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic,” as used herein, refers to a monocyclic or bicyclic ring system. Monocyclic ring systems are exemplified by any 3- or 4-membered ring containing a heteroatom independently selected from oxygen, nitrogen and sulfur; or a 5-, 6 or 7-membered ring containing one, two or three heteroatoms wherein the heteroatoms are independently members selected from nitrogen, oxygen and sulfur. The 5-membered ring has from 0-2 double bonds and the 6- and 7-membered rings have from 0-3 double bonds. Representative examples of monocyclic ring systems include, but are not limited to, azetidinyl, azepinyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another heterocyclic monocyclic ring system. Representative examples of bicyclic ring systems include but are not limited to, for example, benzimidazolyl, benzoazepine, benzothiazolyl, benzothienyl, benzoxazolyl, benzofuranyl, benzopyranyl, benzothiopyranyl, benzodioxinyl, 1,3-benzodioxolyl, cinnolinyl, indazolyl, indolyl, indolinyl, indolizinyl, naphthyridinyl, isobenzofuranyl, isobenzothienyl, isoindolyl, isoindolinyl, isoquinolinyl, phthalazinyl, pyranopyridyl, quinolinyl, quinolizinyl, quinoxalinyl, quinazolinyl, 2,3,4,5-tetrahydro-1H-benzo[c]azepine, 2,3,4,5-tetrahydro-1H-benzo[b]azepine, 2,3,4,5-tetrahydro-1H-benzo[d]azepine, tetrahydroisoquinolinyl, tetrahydroquinolinyl, and thiopyranopyridyl.

The heterocycles of this invention may be optionally substituted with 0, 1, 2 or 3 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, aryl, arylcarbonyl, aryloxy, arylsulfonyl, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, heterocycle, heterocyclecarbonyl, heterocycleoxy, heterocyclesulfonyl, hydroxy, hydroxyalkoxy, hydroxyalkyl, mercapto, mercaptoalkoxy, mercaptoalkyl, methylenedioxy, oxo, nitro, R_{f}R_{g}N—, R_{f}R_{g}Nalkyl, R_{f}R_{g}Ncarbonyl and R_{f}R_{g}Nsulfonyl, wherein R_{f} and R_{g} are members independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “heterocyclealkoxy,” as used herein, refers to a heterocycle, as defined herein, appended to the parent molecular moiety through an alkoxy group, as defined herein.

The term “heterocycleoxy,” as used herein, refers to a heterocycle, as defined herein, appended to the parent molecular moiety through an oxy group, as defined herein.

The term “heterocycleoxyalkyl,” as used herein, refers to a heterocycleoxy, as defined herein, appended to the parent molecular moiety through an alkyl group, as defined herein.

The term “heterocycle-NH—,” as used herein, refers to a heterocycle, as defined herein, appended to the parent molecular moiety through a nitrogen atom.

The term “heterocycle-NH-alkyl,” as used herein, refers to a heterocycle-NH—, as defined herein, appended to the parent molecular moiety through an alkyl group, as defined herein.

The term “heterocycle-heterocycle,” as used herein, refers to a heterocycle, as defined herein, appended to the parent molecular moiety through a heterocycle group, as defined herein.

The term “heterocyclesulfonyl,” as used herein, refers to a heterocycle, as defined herein, appended to the parent molecular moiety through a sulfonyl group, as defined herein. Representative examples of heterocyclesulfonyl include, but are not limited to, 1-piperidinylsulfonyl, 4-morpholinylsulfonyl, pyridin-3-ylsulfonyl and quinolin-3-ylsulfonyl.

The term “non-aromatic,” as used herein, refers to a monocyclic or bicyclic ring system that does not contain the appropriate number of double bonds to satisfy the rule for aromaticity. Representative examples of a “non-aromatic” heterocycles include, but not limited to, piperidinyl, piperazinyl, homopiperazinyl, and pyrrolidinyl. Representative bicyclic ring systems are exemplified by any of the above monocyclic ring systems fused to an aryl group as defined herein, a cycloalkyl group as defined herein, or another heterocyclic monocyclic ring system.

The term “oxo,” as used herein, refers to a ═O group appended to the parent molecule through an available carbon atom.

The term “oxy,” as used herein, refers to a —O— group.

The term “sulfonyl,” as used herein, refers to a —S(O)₂— group.

Salts

The present compounds may exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts may be prepared during the final isolation and purification of the compounds or separately by reacting an amino group of the compounds with a suitable acid. For example, a compound may be dissolved in a suitable solvent such as, but not limited to, methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide the salt.

Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds may also be quaternized with alkyl chlorides, bromides, and iodides such as methyl, ethyl, propyl, isopropyl, butyl, lauryl, myristyl, stearyl, and the like. The present invention also includes pharmaceutically acceptable salts of any compounds of formulas I thru X. In general, salt formation (during the purification of the compounds) is taught in the procedure outlined in Example 8.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

Prodrugs

The present compounds may also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (I-X) for example, by hydrolysis in blood. The term “prodrug,” refers to compounds that contain, but are not limited to, substituents known as “therapeutically suitable esters.” The term “therapeutically suitable ester,” refers to alkoxycarbonyl groups appended to the parent molecule on an available carbon atom. More specifically, a “therapeutically suitable ester,” refers to alkoxycarbonyl groups appended to the parent molecule on one or more available aryl, cycloalkyl and/or heterocycle groups as defined herein. Compounds containing therapeutically suitable esters are an example, but are not intended to limit the scope of compounds considered to be prodrugs. Examples of prodrug ester groups include pivaloyloxymethyl, acetoxymethyl, phthalidyl, indanyl and methoxymethyl, as well as other such groups known in the art. Other examples of prodrug ester groups are found in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. Potential prodrug sites include “therapeutically suitable esters” at the carboxyl group of Example 8 (i.e., alkoxycarbonyl groups in the place of the carboxyl group).

Optical Isomers-Diastereomers-Geometric Isomers

Asymmetric centers may exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers may exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

### Preparation of Compounds of The Invention

The compounds and processes of the present invention will be better understood in connection with the following synthetic schemes and Experimentals that illustrate a means by which the compounds of the invention may be prepared.

The compounds of this invention may be prepared by a variety of procedures and synthetic routes. Representative procedures and synthetic routes are shown in, but are not limited to, Schemes 1-3.

Abbreviations which have been used in the descriptions of the Schemes and the Examples that follow are: DCM for dichloromethane; DMAP for dimethylaminopyridine; DMF for N,N-dimethylformamide; DMSO for dimethylsulfoxide; DAST for (diethylamino)sulfur trifluoride; DIPEA or Hunig's base for diisopropylethylamine; DMA for dimethylacetamide; EDCI for (3-dimethylaminopropyl)-3-ethylcarbodiimide HCl; EtOAc for ethyl acetate; EtOH for ethanol; HATU for O-(7-azabenzotriazol-1-yl)-N,N,N′,N′-tetramethyluronium hexafluoro-phosphate; HOAc for acetic acid; HOBt for hydroxybenzotriazole hydrate; MeOH for methanol; mesyl for methanesulfonyl; TEA for triethylamine; TFA for trifluoroacetic acid; THF for tetrahydrofuran; tosyl for para-toluenesulfonyl; triflate for trifluoromethanesulfonyl.

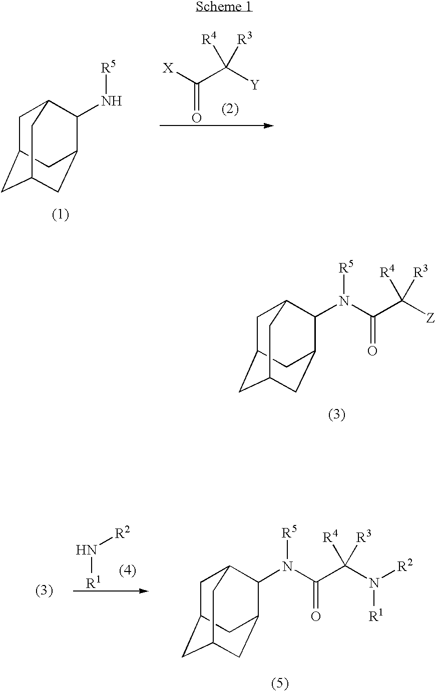

Adamantanes of general formula (5), wherein R¹, R², R³, R⁴, and R⁵ are as defined in formula I, may be prepared as in Scheme 1. 2-adamantamine and related amines of general formula (1) may be purchased or prepared by methods known to those in the art. For instance 2-adamantamine may undergo reductive amination with an aldehyde or ketone. Amines of general formula (1) may be treated with acylating agents such as chloroacetyl chloride or 2-bromopropionyl bromide of general formula (2), wherein X is Cl, Br, or F, R³ and R⁴ are defined as in formula I, and Y is a leaving group like Cl or Br (or a protected or masked leaving group), and a base such as diisopropylethylamine to provide amides of general formula (3). Alternatively, acids of general formula (2), wherein X is OH, may be coupled to an amine of general formula (1) like 2-adamantamine with reagents such as EDCI and HOBt to provide amides of general formula (3). When Y is a leaving group like chlorine or bromine, Y equals Z. When Y is a protected or masked leaving group, Y is converted into Z where Z is a leaving group like Cl, Br, I, —O-tosyl, —O-mesyl, or —O-triflate after amide formation. Amides of general formula (3) may be treated with amines of general formula (4) wherein R¹ and R² are as defined in formula I to provide aminoamides of general formula (5).

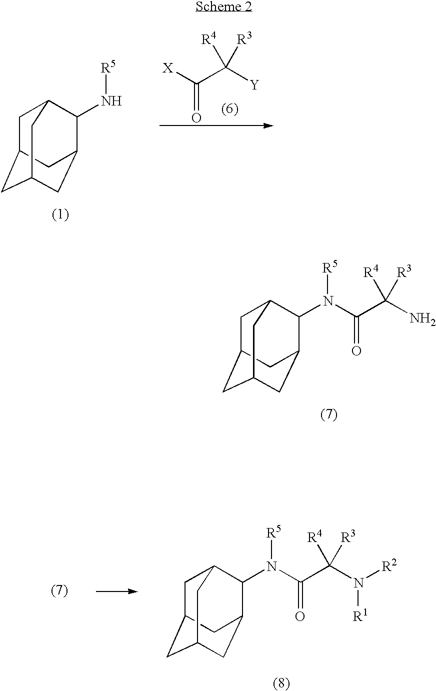

Adamantanes of general formula (8), wherein R¹, R², R³, R⁴, and R⁵ are as defined in formula I, may be prepared as in Scheme 2. 2-adamantamine and related amines of general formula (1) may be purchased or prepared by methods known to those in the art. For instance 2-adamantamine may undergo reductive amination with an aldehyde or ketone. Amines of general formula (1) may be coupled with protected amino acids of general formula (6), wherein X is OH, R³ and R⁴ are defined as in formula I, and Y is a protected or masked amine, such as N-(tert butoxycarbonyl)glycine with reagents such as EDCI and HOBt to provide amides of general formula (7) after deprotection. Alternatively, amines of general formula (1) may be treated with activated protected amino acids of general formula (6), wherein X is Cl, Br, or F, and a base such as diisopropylethylamine to provide amides of general formula (7) after deprotection. Amides of general formula (7) may be treated with alkylating agents such as 1,5-dibromopentane and a base like potassium carbonate to yield amides of general formula (8). Among other methods known to those in the art, amides of general formula (7) may be treated with aldehydes such as benzaldehyde and a reducing agent like sodium cyanoborohydride to yield amides of general formula (8).

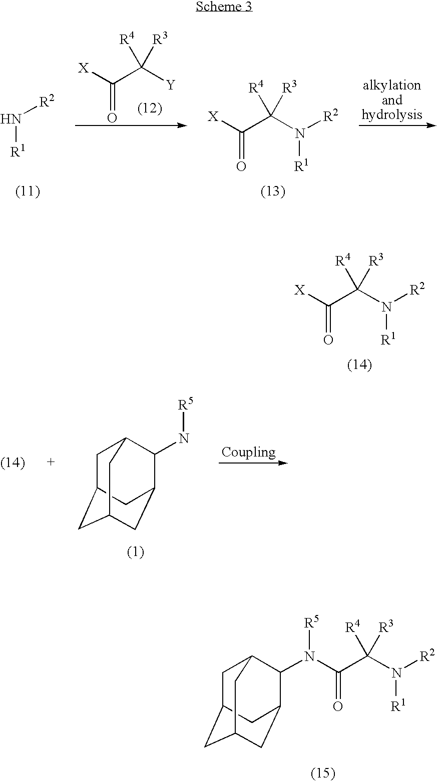

Adamantanes of general formula (15), wherein R¹, R², R³, R⁴, and R⁵ are as defined in formula I, may be prepared as in Scheme 3. Amines of general formula (11) may be purchased or prepared using methodology known to those in the art. The amines of general formula (11) may be reacted with reagents of general formula (12), wherein R³ and R⁴ are defined as in formula I, Y is a leaving group such as Cl, Br, I, —O-tosyl, —O-mesyl, or —O-triflate, and X is an alkoxy group, such as 2-bromopropionic acid methyl ester in the presence of a base like diisopropylethylamine to provide esters of general formula (13). Esters of general formula (13) may be alkylated using a base like lithium diisopropylamide and an alkylating agent such as methyl iodide to yield acids of general formula (14), X═OH, after hydrolysis. Amines of general formula (1) may be coupled to acids of general formula (14) with reagents such as EDCI and HOBt to provide amides of general formula (15).

The compounds and processes of the present invention will be better understood by reference to the following Examples, which are intended as an illustration of and nota limitation upon the scope of the invention. Further, all citations herein are incorporated by reference.

Compounds of the invention were named by ACD/ChemSketch version 5.01 (developed by Advanced Chemistry Development, Inc., Toronto, ON, Canada) or were given names consistent with ACD nomenclature.

### EXAMPLE 1

### N-2-adamantyl-2-[4-(5-chloropyridin-2-yl)piperazin-1-yl]acetamide

### EXAMPLE 1A

### N-Adamantan-2-yl-2-chloro-acetamide

A solution of 2-adamantamine hydrochloride (1.8 g, 9.6 mmoles) and diisopropylethylamine (DIPEA) (3.48 mL, 20 mmoles) in DCM (30 mL) was cooled in an ice bath and treated with chloroacetyl chloride (0.78 mL, 9.65 mmoles). The solution was stirred for 2 hours at room temperature and the DCM was removed under reduced pressure. The residue was partitioned between water and ethyl acetate. The organic layer was washed with saturated sodium bicarbonate and with water, dried over MgSO₄ and filtered. The filtrate was concentrated under reduced pressure to provide the title compound as a dark tan solid (2.1 g, 92.5%).

### EXAMPLE 1B

### 4-(Adamantan-2-ylcarbamoylmethyl)-piperazine-1-carboxylic acid tert-butyl ester

N-Adamantan-2-yl-2-chloro-acetamide (5.2 g, 22.8 mmoles) from Example 1A, piperazine-1-carboxylic acid tert-butyl ester (5.32 g, 28.5 mmoles), and triethylamine (4.0 mL, 28.5 mmoles) were added to a room temperature solution of CH₃CN (23 mL) and THF (23 mL). After stirring for 48 h the reaction was concentrated and chromatographed on silica gel (4:1→1:4 hexane:EtOAc) to provide the title compound (5.44 g, 63%).

### EXAMPLE 1C

### N-Adamantan-2-yl-2-piperazin-1-yl-acetamide

4-(Adamantan-2-ylcarbamoylmethyl)-piperazine-1-carboxylic acid tert-butyl ester (5.4 g, 14.3 mmoles) from Example 1B was dissolved in CH₂Cl₂ (34 mL) and TFA (7 mL) and stirred at room temperature for 4 hours. The mixture was concentrated in vacuo, toluene (50 mL) was added, and the resulting mixture concentrated in vacuo again to provide a crude sample of the bis(trifluoroacetic acid) salt of the title compound.

### EXAMPLE 1D

### N-2-adamantyl-2-[4-(5-chloropyridin-2-yl)piperazin-1-yl]acetamide

A solution of the bis(trifluoroacetic acid) salt of N-adamantan-2-yl-2-piperazin-1-yl-acetamide (51 mg, 0.1 mmoles), from Example 1C, in dimethylsulfoxide (DMSO) (0.33 mL) and 2N aqueous sodium carbonate (0.2 mL) was treated with 2,5-dichloro-pyridine (30 mg, 0.2 mmoles) and irradiated by microwaves for 20 min at 240° C. The reaction mixture was filtered through a Celite cartridge and purified by HPLC to provide the title compound as a white solid (20 mg, 50%). ¹H NMR (300 MHz, CDCl₃) δ 8.12 (d, J=2.5 Hz, 1H), 7.73 (d, J=8.8 Hz, 1H), 7.44 (dd, J=2.5 Hz, 9.2 Hz, 1H), 6.61 (d, J=9.2 Hz, 1H), 4.10 (d, J=8.9 Hz, 1H), 3.56 (t, J=5 Hz, 4H), 3.12 (s, 2H), 2.69 (t, J=5 Hz, 4H), 1.91 (s, 2H), 1.87 (d, J=1.9 Hz, 6H), 1.75 (m, 4H), 1.67 (m, 2H); MS (APCI+) m/z 389 (M+H)⁺.

### EXAMPLE 2

### N-2-adamantyl-2-[4-(5-chloropyridin-2-yl)piperazin-1-yl]propanamide

### EXAMPLE 2A

### 2-Chloro-N-adamantan-2-yl-propionamide

A solution of 2-adamantamine hydrochloride (1.87 g, 10 mmoles) in DCM (30 mL) and DIPEA (4.16 mL, 24 mmoles) was cooled in an ice bath and treated with 2-chloropropionyl chloride (0.93 mL, 11 mmoles). The solution was stirred for 2 hours at room temperature and DCM was removed under reduced pressure. The residue was partitioned between water and ethyl acetate. The organic layer was washed with saturated sodium bicarbonate and with water, dried over MgSO₄ and filtered. The filtrate was concentrated under reduced pressure to provide the title compound as a dark tan solid (2.2 g, 92.3%).

### EXAMPLE 2B

### 4-[1-(Adamantan-2-ylcarbamoyl)-ethyl]-piperazine-1-carboxylic acid tert-butyl ester

A solution of 2-chloro-N-adamantan-2-yl-propionamide (2.4 g, 10 mmoles), from Example 2A, in dimethylformamide (DMF) (33 mL) and 2N aqueous sodium carbonate (15 mL) was treated with Boc-piperazine (1.86 g, 10 mmoles). The solution was stirred overnight at 60° C. and DMF was removed under reduced pressure. The residue was partitioned between water and ethyl acetate. The organic layer was washed twice with water, dried over MgSO₄ and filtered. The filtrate was concentrated under reduced pressure to provide the title compound as a white solid (2.9 g, 74.3%).

### EXAMPLE 2C

### N-Adamantan-2-yl-2-piperazin-1-yl-propionamide hydrochloride

4-[1-(Adamantan-2-ylcarbamoyl)-ethyl]-piperazine-1-carboxylic acid tert-butyl ester (2.9 g, 7.4 mmoles), from Example 2B, was dissolved in a 4N HCl solution in dioxane (50 mL). The resulting solution was stirred for 4 hours at room temperature. Dioxane was removed under reduced pressure to provide a bis(hydrochloride) salt of the title compound as a white solid (2.4 g, 99%)

### EXAMPLE 2D

### N-2-Adamantyl-2-[4-(5-chloropyridin-2-yl)piperazin-1-yl]propanamide

A solution of the bis(hydrochloride) salt of N-adamantan-2-yl-2-piperazin-1-yl-propionamide (37 mg, 0.1 mmoles), from Example 2C, in dimethylsulfoxide (DMSO) (0.33 mL) and 2N aqueous sodium carbonate (0.2 mL) was treated with 2,5-dichloro-pyridine (30 mg, 0.2 mmoles) and irradiated by microwaves for 20 min at 240° C. The reaction mixture was filtered through a Celite cartridge and purified by HPLC to provide the title compound as a white solid (20 mg, 50%). ¹H NMR (300 MHz, CDCl₃) δ 8.12 (d, J=2.8 Hz, 1H), 7.76 (d, J=8.5 Hz, 1H), 7.44 (dd, J=2.5, 9.2 Hz, 1H), 6.61 (d, J=9.2 Hz, 1H), 4.05 (d, J=8.5 Hz, 1H), 3.54 (s, 4H), 3.12 (d, J=6.5 Hz, 1H), 2.68 (m, 4H), 1.89 (m, 8H), 1.75 (s, 4H), 1.67(m, 2H), 1.28 (d, J=6.7 Hz, 3H); MS (APCI+) m/z 403 (M+H)⁺.

### EXAMPLE 3

### N-2-Adamantyl-2-{4-[2-(benzyloxy)ethyl]piperazin-1-yl }acetamide

Library synthesis was performed using a PE Biosystems (Applied Biosystems) Solaris 530 organic synthesizer. All monomers used in the automated synthesis were stored under inert atmosphere and supplied as either oils or solids in capped 4 mL Kimble vials (Kimble 60881 A-1545) from Aldrich Chemical Co. Other reagents were used directly as obtained from the manufacturer. Each of the 48 round bottom flasks was charged with 3 equivalents of PS—BH₃CN resin (Argonaut Technologies). The reaction block was then assembled, placed on the Solaris 530 and purged with nitrogen for 45 seconds. The alcohol monomers (0.6 mmoles) were each dissolved in 3 mL of DMA and the HOAc and amine core were each dissolved in 17 and 10 mL of 50/50 MeOH/DCM, respectively, and placed on the instrument. To the monomer solutions was added 0.5 mmoles of Dess-Martin periodinane reagent (Aldrich Chemical Co.). The monomer/Dess-Martin periodinane solution was shaken at room temperature for 30 minutes. The Solaris was then primed with MeOH and into each of the 48 flasks containing PS—BH₃CN resin was added 0.75 mL of the core solution (1 eq.) followed by 0.75 mL of HOAc solution (1 eq) and 1.5 eq of each monomer solution. The reactions were heated to 55° C. overnight, checked by LC/MS to confirm that the transformations were complete, filtered and transferred to 20 mL vials containing 3 eq. of MP-Carbonate and 2 eq. of PS-TsNHNH₂ (Argonaut Technologies) resin. The reaction vessels and PS—BH₃CN resin were washed with MeOH and the combined filtrates were shaken over the MP-carbonate/PS-TsNHNH₂ resins for 2 hours at room temperature. The MP-Carbonate/PS-TsNHNH₂ resins were removed via filtration and the reactions were concentrated to dryness. The residues were dissolved in 1:1 DMSO/MeOH (1.2 mL) and purified by reverse-phase HPLC. The monomer in this case was 2-benzyloxy-ethanol and the core was the product of Example 1C. ¹H NMR (500 MHz, pyridine-d₅) δ ppm 1.59 (d, J=12.2 Hz, 2 H) 1.65 (s, 2 H) 1.74 (m, 7 H) 1.89 (d, J=12.8 Hz, 2 H) 1.98 (m, J=4.7 Hz, 2 H) 2.59 (m, 7 H) 2.66 (t, J=5.9 Hz, 2 H) 3.16 (s, 2 H) 3.65 (m, 2 H) 4.29 (m, 1 H) 4.56 (s, 2 H) 7.31 (t, J=7.95 Hz, 1 H) 7.39 (m, J=7.49, 7.5 Hz, 3 H) 7.47 (d, J=6.9 Hz, 2 H); MS(ESI) positive ion 412.1 (M+H)⁺.

### EXAMPLE 4

### N-2-Adamantyl-2-[4-(2-furoyl)piperazin-1-yl]propanamide

A solution of 2-chloro-N-adamantan-2-yl-propionamide (48 mg, 0.2 mmoles), from Example 2A, in dimethylformamide (DMF) (0.5 mL) and 2N aqueous sodium carbonate (0.1 mL) was treated with furan-2-yl-piperazin-1-yl-methanone. The solution was stirred overnight at 70° C. and DMF was removed under reduced pressure. The residue was partitioned between water and ethyl acetate. The organic layer was washed twice with water, dried over MgSO₄ and filtered. The filtrate was concentrated under reduced pressure and purified by HPLC to provide the title compound as a white solid (43 mg, 56%). ¹H NMR (300 MHz, CDCl₃) δ 7.67 (d, J=8.5 Hz, 1H), 7.48 (s, 1H), 7.01 (d, J=3.4 Hz, 1H), 6.48 (q, J=1.5, 3.4 Hz, 1H), 4.05 (d, J=8.7 Hz, 1H), 3.84 (s, 4H), 3.12(q, J=7.2 Hz, 1H), 2.63 (m, 4H), 1.9-1.86 (m, 8H), 1.76-1.68 (m, 6H), 1.26 (d, 7.2, 3H); MS (APCI+) m/z 386 (M+H)⁺.

### EXAMPLE 5

### N-2-Adamantyl-2-(4-hydroxypiperidin-1-yl)propanamide

All monomers used in the synthesis were stored under inert atmosphere and supplied as either oils or solids in capped 4 mL Kimble vials (Kimble 60881A-1545) from Aldrich Chemical Co. Other reagents were used directly as obtained from the manufacturer. The core was dissolved in 72 ml of 50/50 MeOH/DMSO and 1.5 mL of the core solution was added to each 4 mL vial containing (0.6 mmoles, 6.8 eq.) of amine monomer. The reactions were heated to 70° C. overnight and checked by LC/MS to confirm that the transformations were complete. The reactions were concentrated to dryness. The residues were dissolved in 1:1 DMSO/MeOH (1.2 mL) and purified by reverse-phase HPLC. The monomer in this case was 4-hydroxypiperidine and the core was the product of Example 2A. ¹H NMR (500 MHz, CDCl₃) δ ppm 1.54 (d, J=6.86 Hz, 3 H) 1.61 (d, J=12.48 Hz, 2 H) 1.74 (s, 2 H) 1.85 (m, 10 H) 2.00 (s, 1 H) 2.24 (s, 2 H) 3.23 (s, 1 H) 3.36 (s, 2 H) 3.60 (m, 1 H) 4.03 (m, 1 H) 4.19 (m, 2 H) 7.80 (m, 1 H); MS (ESI) positive ion 307.0 (M+H)⁺.

### EXAMPLE 7

### N-2-Adamantyl-1-(pyridin-2-ylmethyl)piperidine-2-carboxamide

### EXAMPLE 7A

### 2-(Adamantan-2-ylcarbamoyl)-piperidine-1-carboxylic acid benzyl ester

1-(Benzyloxycarbonyl)-piperidine-2-carboxylic acid [M. J. Genin, W. B. Gleason, R. L. Johnson J. Org. Chem. 1993, 58 (4), 860-866], (5.26 g., 0.02 mol) and diisopropyethylamine (3.10 g, 0.024 mol) were dissolved in 35 mL. dichloromethane. 1-Hydroxybenzotriazole (3.366 g., 0.022 mol) was added. When all of the solids dissolved, 2-amino-adamantane HCl (4.50 g., 0.024 mol) was added. Finally, EDCI.HCl (4.60 g., 0.024 mol) was added. After stirring 10 minutes, a clear solution was observed. After stirring 18 hours at room temperature, the solution was concentrated under reduced pressure and toluene was added. The organic phase was washed with aqueous Na₂CO₃, water, dilute HCl, and then aqueous KHCO₃. After drying over Na₂SO₄, the solvents were removed in vacuum to yield the title compound (6.65 g, 84% yield). TLC in ethyl acetate was one spot, Rf=0.65.

### EXAMPLE 7B

### Piperidine-2-carboxylic acid adamantan-2-ylamide

The product of Example 7A (6.55 g., 16.52 mmoles) was dissolved in methanol (125 mL). 10% Pd on carbon (665 mg.) was added and the mixture was hydrogenated with 4 atmospheres H₂ at room temperature for 1 hour. The catalyst was removed by filtration, and the solution concentrated under reduced pressure. Heptane was added and removed under reduced pressure (3 times). The residue was crystallized from ether and heptane (1:3) to provide the title compound (4.33 g, 100%, mp 112-114° C.).

### EXAMPLE 7C

### N-2-Adamantyl-1-(pyridin-2-ylmethyl)piperidine-2-carboxamide

The product of Example 7B (263 mg., 1.0 mm.) and diisopropylethylamine (387 mg, 3.0 mmoles) were dissolved in DMF (1.5 mL). 2-(Chloromethyl)-pyridine HCl (175 mg, 1.067 mmoles) was added. The mixture was stirred for 5 hours at room temperature. Toluene and aqueous KHCO₃ were added and shaken. The toluene phase was dried (Na₂SO₄) and the solution concentrated under reduced pressure. The residue was chromatographed on silica gel, eluting with 5% methanol in dichloromethane to yield the title compound (211 mg, mp 126-127° C.). NMR (300 MHz, CDCl₃) 1.15-1.20 (m, 1H), 1.22-1.98 (m, 19H), 2.03-2.17 (m, 2H), 2.85-2.95 (m, 2H), 3.35 (d, J=13 Hz, 1H), 4.01 (d, J=13 Hz, 1H), 4.15 (s, 1H), 7.15 (dd, J=4 Hz, J=2 Hz, 1H), 7.24 (d, J=7 Hz), 1H), 7.63 (dt, J=7 Hz, J=2 Hz, 1H), 7.68 (s, 1H), 8.55 (dd, J=4 Hz, J=1 Hz, 1H).

### EXAMPLE 8

### 4-({2-[(2-Adamantylamino)carbonyl]pyrrolidin-1-yl]methyl)benzoic acid

A stirred solution of pyrrolidine-2-carboxylic acid adamantan-2-ylamide trifluoracetic acid salt (73 mg, 0.2 mmoles) from Example 6C,N,N-diisoproylethylamine (52 mg, 0.4 mmoles), 4-bromomethyl-benzoic acid (43 mg, 0.2 mmoles), dimethylsulfoxide (1.5 mL) and methanol (1.5 mL) was heated to 70° C. for 18 hours. The mixture was cooled to 23° C. and purified by preparative HPLC on a Waters Symmetry C8 column (40 mm×100 mm, 7 μm particle size) using a gradient of 10% to 100% acetonitrile: 0.1% aqueous TFA over 12 min (15 min run time) at a flow rate of 70 mL/min to afford the trifluoroacetic acid salt of the title compound (51.6 mg, 51%) upon concentration in vacuo. ¹H NMR (300 MHz, DMSO-d₆) δ 13.10 (bs, 1H), 9.66 (bs, 1H), 8.15 (m, 1H), 7.93 (d, J=8.4 Hz, 2H), 7.58 (d, J=8.1 Hz, 2H), 4.48 (m, 1H), 4.38 (m, 1H), 4.19 (m, 1H), 3.61 (m, 1H), 2.07 (m, 1H), 1.70 (m, 16H), 1.27 (m, 3H); MS (DCI) m/z 383 (M+H)⁺.

### EXAMPLE 9

### N-2-Adamantyl-1-[4-(aminocarbonyl)benzyl]prolinamide

A 0° C. heterogenous solution of 4-[2-(adamantan-2-ylcarbamoyl)-pyrrolidin-1-ylmethyl]-benzoic acid (50 mg, 0.13 mmoles) from Example 8 and CH₂Cl₂ (6 mL) was treated with oxalyl chloride (20 mg, 0.16 mmoles) and catalytic N,N-dimethylformamide. The reaction mixture was slowly warmed to 23° C. and remained heterogeneous even after 2 hours. To the reaction mixture was added tetrahydrofuran (4 mL) and thionyl chloride (0.5 mL), and the reaction temperature raised to reflux for 30 minutes. The reaction mixture was cooled to 23° C., concentrated under reduced pressure, and re-dissolved in tetrahydrofuran (1 mL). To this stirred reaction mixture at 23° C. was added 0.5 M NH₃ in dioxane (1.05 mL, 0.55 mmoles) followed after 30 min by H₂O (0.25 mL). After another 30 min, the reaction mixture was concentrated under reduced pressure and purified by preparative HPLC on a Waters Symmetry C8 column (40 mm×100 mm, 7 μm particle size) using a gradient of 10% to 100% acetonitrile: ammonium acetate (10 mM) over 12 minutes (15 minute run time) at a flow rate of 70 mL/min to afford the title compound (11 mg, 22%). ¹H NMR (300 MHz, DMSO-d₆) δ 7.92 (bs, 1H), 7.83 (d, J=8.1 Hz, 2H), 7.73 (d, J=8.4 Hz, 1H), 7.38 (d, J=8.1 Hz, 2H), 7.31 (bs, 1H), 3.86 (d, J=13.8 Hz, 1H), 3.77 (d, J=8.4 Hz, 1H), 3.59 (d, J=13.5 Hz, 1H), 3.16 (dd, J=4.8, 9.9 Hz, 1H), 2.98 (m, 1H), 2.36 (m, 1H), 2.10 (m, 1H), 1.72 (m, 15H), 1.54 (m, 2H); MS (DCI) m/z 382 (M+H)⁺.

### EXAMPLE 10

### N-2-Adamantyl-2-methyl-2-piperidin-1-ylpropanamide

### EXAMPLE 10A

### [1-(Adamantan-2-ylcarbamoyl)-1-methyl-ethyl]-carbamic acid tert-butyl ester

To a solution of 2-tert-butoxycarbonylamino-2-methyl-propionic acid (1.0 g, 4.9 mmoles) and CH₂Cl₂ (45 mL) cooled to 0° C. was added in order 1-hydroxybenzotriazole hydrate (0.62 g, 4.9 mmoles), 1-(3-dimethylaminopropyl)-3-ethylcarbodiimide hydrochloride (0.94 g, 4.9 mmol), and triethylamine (2.2 mL, 16 mmoles). After 10 minutes 2-aminoadamantane hydrochloride (1.0 g, 5.4 mmoles) was added to the reaction mixture. The reaction temperature was maintained at 0° C. another 15 minutes and then warmed to 23° C. for 16 hours. The reaction mixture was partitioned between aqueous 10% Citric acid and additional CH₂Cl₂. The layers were separated and the aqueous layer extracted twice more with CH₂Cl₂. The combined CH₂Cl₂ layers were washed similarly with aqueous saturated NaHCO₃ and brine solutions before drying over Na₂SO₄, filtration, and concentration under reduced pressure to afford a crude sample of the title compound (1.48 g, 90%).

### EXAMPLE 10B

### N-Adamantan-2-yl-2-amino-2-methyl-propionamide

To a 0° C. solution of a crude sample of the product of Example 10A (0.5 g, 1.5 mmoles) in CH₂Cl₂ (10 mL) was added trifluoroacetic acid (1.14 mL) and upon completion of addition, the cooling bath was removed. The reaction mixture was stirred at 23° C. for 4 hours. The reaction mixture was concentrated under reduced pressure and azeotroped with 5:1 toluene/methanol (3×5 mL) to afford a sample of the crude title compound as its trifluoroacetic acid salt (0.52 g, 100%).

### EXAMPLE 10C

### N-2-Adamantyl-2-methyl-2-piperidin-1-ylpropanamide

A sealed tube containing the product of Example 10B (0.13 g, 0.37 mmoles), 1,5-dibromopentane (0.17 g, 0.74 mmoles), K₂CO₃ (0.21 g, 1.5 mmoles), and ethanol (2 mL) was rapidly stirred and heated to 90° C. in an oil bath for 18 h. The reaction mixture was cooled, filtered, and concentrated under reduced pressure. The residue was purified by chromatography (flash silica gel, 20-100% ethyl acetate in hexanes) to afford the title compound (26 mg, 23%). ¹H NMR (300 MHz, DMSO-d₆) δ 7.78 (d, J=8.4 Hz, 1H), 3.77 (d, J=8.1 Hz, 1H), 2.38 (bs, 4H), 1.77 (bm, 12H), 1.51 (bm, 8H), 1.07 (s, 6H); MS (DCI) m/z 305 (M+H)⁺.

### EXAMPLE 11

### N-2-Adamantyl-2-methyl-2-{4-[5-(trifluoromethyl)pyridin-2-yl]piperazin-1-yl}propanamide

### EXAMPLE 11A

### 2-[4-(5-Trifluoromethyl-pyridin-2-yl)-piperazin-1-yl]-propionic acid methyl ester

A solution of 1-(5-trifluoromethyl-pyridin-2-yl)-piperazine (0.9 g, 3.9 mmoles) in MeOH (13 mL) and DIPEA (1.5 mL) was treated with 2-bromo-propionic acid methyl ester (0.48 mL, 4.3 mmoles) and stirred overnight at 70° C. MeOH was removed under reduced pressure and the residue was purified (silica gel, 10-40% acetone in hexane) to provide the title compound as a yellowish solid (1.23 g, 99%).

### EXAMPLE 11B

### 2-Methyl-2-[4-(5-trifluoromethyl-pyridin-2-yl)-piperazin-1-yl]-propionic acid methyl ester

A solution of 2-[4-(5-trifluoromethyl-pyridin-2-yl)-piperazin-1-yl]-propionic acid methyl ester (1.23 g, 3.9 mmoles), from Example 11A, in dry THF (3 mL) was added dropwise to a −65° C. solution of 1.8 N lithium diisopropylamine (LDA) in dry THF (2.4 mL) and stirred at this temperature for 1 hour. Methyl iodide (0.49 mL, 7.88 mmoles) was then added to the reaction mixture. The reaction was allowed to slowly warm to room temperature and stir for 2 hours at room temperature. The reaction was quenched with ice/water and partitioned between water and ethyl acetate. The aqueous layer was extracted with ethyl acetate. The combined organic extracts were washed with water, dried over MgSO₄, filtered and the filtrate concentrated under reduced pressure. The residue was purified (silica gel, 10-30% acetone in hexane) to provide the title compound as a yellowish solid (1.05 g, 81.7%)

### EXAMPLE 11C

### 2-Methyl-2-[4-(5-trifluoromethyl-pyridin-2-yl)-piperazin-1-yl]-propionic acid

A solution of 2-methyl-2-[4-(5-trifluoromethyl-pyridin-2-yl)-piperazin-1-yl]-propionic acid methyl ester (1.05 g, 3.17 mmoles), from Example 11B, in dioxane (10 mL) was treated with 5N aqueous potassium hydroxide (10 mL) and stirred for 4 hours at 60° C. Dioxane was removed under reduced pressure, the residue neutralized with 1N HCl to pH=7 and extracted three times with 4:1 THF:DCM. The combined organic extracts were dried over MgSO₄, filtered and the filtrate concentrated under reduced pressure to provide the title compound as a white solid (0.9 g, 90%)

### EXAMPLE 11D

### N-2-Adamantyl-2-methyl-2-{4-[5-(trifluoromethyl)pyridin-2-yl]piperazin-1-yl}propanamide

A solution of 2-methyl-2-[4-(5-trifluoromethyl-pyridin-2-yl)-piperazin-1-yl]-propionic acid (159 mg, 0.5 mmoles), from Example 11C, in DCM (5 mL) and DIPEA (0.5 mL) was treated with hydroxybenzotriazole hydrate (HOBt) (84 mg, 0.6 mmoles), 2-adamantamine hydrochloride (112 mg, 0.6 mmoles) and 15 min later with (3-dimethylaminopropyl)-3-ethylcarbodiimide HCl (EDCI) (115 mg, 0.6 mmoles). The reaction mixture was stirred overnight at room temperature. DCM was removed under reduced pressure and the residue was partitioned between water and ethyl acetate. The aqueous layer was extracted with ethyl acetate. The combined organic extracts were washed with saturated aqueous sodium bicarbonate and water, dried over MgSO₄ and filtered. The filtrate was concentrated under reduced pressure and the crude product purified (silica gel, 10-40% acetone in hexane) to provide the title compound as a white solid (160 mg, 69%). ¹H NMR (300 MHz, CDCl₃) δ 8.41 (s, 1H), 7.79 (d, J=6.5 Hz, 1H), 7.65 (m, 1H), 6.66 (d, J=9.2 Hz, 1H), 4.02 (d, J=6.8 Hz, 1H), 3.66 (m, 4H), 2.65 (t, J=5.1 Hz, 4H), 1.9-1.86 (m, 8H), 1.75-1.69 (m, 6H), 1.24 (s, 6H); MS(APCI+) m/z 451 (M+H)⁺.

### Biological Data

Measurement of Inhibition Constants:

The ability of test compounds to inhibit human 11-βHSD-1 enzymatic activity in vitro was evaluated in a Scintillation Proximity Assay (SPA). Tritiated-cortisone substrate, NADPH cofactor and titrated compound were incubated with truncated human 11β-HSD-1 enzyme (24-287AA) at room temperature to allow the conversion to cortisol to occur. The reaction was stopped by adding a non-specific 11β-HSD inhibitor, 18β-glycyrrhetinic acid. The tritiated cortisol generated was then captured by a mixture of an anti-cortisol monoclonal antibody and SPA beads coated with anti-mouse antibodies. The reaction plate was shaken at room temperature and the radioactivity bound to SPA beads was then measured on a β-scintillation counter. The 11-βHSD-1 assay was carried out in 96-well microtiter plates in a total volume of 220 μl. To start the assay, 188 μl of master mix which contains 17.5 nM ³H-cortisone, 157.5 nM cortisone, and 181 mM NADPH was added to the wells. In order to drive the reaction in the forward direction, 1 mM G-6P was also added. Solid compound was dissolved in DMSO to make a 10 mM stock followed by a subsequent 10-fold dilution with 3% DMSO in Tris/EDTA buffer (pH 7.4). 22 μl of titrated compounds was then added in triplicate to the substrate. Reactions were initiated by the addition of 10 μl of 0.1 mg/ml E.coli lysates overexpressing 11β-HSD-1 enzyme. After shaking and incubating plates for 30 minutes at room temperature, reactions were stopped by adding 10 μl of 1 mM glycyrrhetinic acid. The product, tritiated cortisol, was captured by adding 10 μl of 1 μM monoclonal anti-cortisol antibodies and 100 μl SPA beads coated with anti-mouse antibodies. After shaking for 30 minutes, plates were read on a liquid scintillation counter Topcount. Percent inhibition was calculated based on the background and the maximal signal. Wells that contained substrate without compound or enzyme were used as the background, while the wells that contained substrate and enzyme without any compound were considered as maximal signal. Percent of inhibition of each compound was calculated relative to the maximal signal and IC₅₀ curves were generated. This assay was applied to 11β-HSD-2 as well, whereby tritiated cortisol and NAD⁺ were used as substrate and cofactor, respectively.

As shown in Table 1, compounds of the present invention are active in the 11-βHSD-1 assay described above and show selectivity for human 11-β-HSD-1 over human 11-β-HSD-2.

TABLE 1

| Example | 11-β-HSD-1 IC₅₀ (nM) | 11-β-HSD-2 IC₅₀ (nM) |
|---|---|---|
| 3 | 35 | — |
| 4 | 46 | — |
| 5 | 34 | >10,000 |
| 7 | 48 | — |

The data in Table I indicates that the compounds of the present invention are active in the human 11β-HSD-1 enzymatic SPA assay described above and show selectivity for 11β-HSD-1 over 11β-HSD-2. The 11β-HSD-1 inhibitors generally have an inhibition constant IC₅₀ of less than 600 nM, and more preferably less than 50 nM. Preferably, the compounds are selective and have an inhibition constant IC₅₀ against 11β-HSD-2 greater than 1000 nM, and more preferably greater than 10,000 nM. Generally, the IC₅₀ ratio for 11β-HSD-2 to 11β-HSD-1 of a compound is at least 10 or greater, and preferably 100 or greater.

The compounds are selective inhibitors of the 11β-HSD-1 enzyme. Their utility in treating or prophylactically treating type 2 diabetes, high blood pressure, dyslipidemia, obesity and other diseases and conditions is believed to derive from the biochemical mechanism described below.

Biochemical Mechanism

Glucocorticoids are steroid hormones that play an important role in regulating multiple physiological processes in a wide range of tissues and organs. For example, glucocorticoids are potent regulators of glucose and lipid metabolism. Excessive glucocorticoid action may lead to insulin resistance, type 2 diabetes, dyslipidemia, visceral obesity and hypertension. Cortisol is the major active and cortisone is the major inactive form of glucocorticoids in humans, while corticosterone and dehydrocorticosterone are the major active and inactive forms in rodents.

Previously, the main determinants of glucocorticoid action were thought to be the circulating hormone concentration and the density of receptors in the target tissues. In the last decade, it was discovered that tissue glucocorticoid levels may also be controlled by 11β-hydroxysteroid dehydrogenases enzymes (11β-HSDs). There are two 11β-HSD isozymes which have different substrate affinities and cofactors. The 11β-hydroxysteroid dehydrogenases type 1 enzyme (11β-HSD-1) is a low affinity enzyme with Kₘ for cortisone in the micromolar range that prefers NADPH/NADP⁺ (nicotinamide adenine dinucleotide) as cofactors. 11β-HSD-1 is widely expressed and particularly high expression levels are found in liver, brain, lung, adipose tissue, and vascular smooth muscle cells. In vitro studies indicate that 11β-HSD-1 is capable of acting both as a reductase and a dehydrogenase. However, many studies have shown that it is a predominant reductase in vivo and in intact cells. It converts inactive 11-ketoglucocorticoids (cortisone or dehydrocorticosterone) to active 11-hydroxyglucocorticoids (cortisol or corticosterone), and therefore amplifies the glucocorticoid action in a tissue-specific manner.

With only 20% homology to 11β-HSD-1, the 11β-hydroxysteroid dehydrogenase type 2 enzyme (11β-HSD-2) is a NAD⁺-dependent, high affinity dehydrogenase with a Kₘ for cortisol in the nanomolar range. 11β-HSD-2 is found primarily in mineralocorticoid target tissues, such as kidney, colon, and placenta. Glucocorticoid action is mediated by the binding of glucocorticoids to receptors, such as mineralocorticoid receptors and glucocorticoid receptors. Through binding to its receptor, the main mineralocorticoid aldosterone controls the water and salts balance in the body. However, the mineralocorticoid receptors have a high affinity for both cortisol and aldosterone. 11β-HSD-2 converts cortisol to inactive cortisone, therefore preventing the non-selective mineralocorticoid receptors from exposure to high levels of cortisol. Mutations in the gene encoding 11β-HSD-2 cause Apparent Mineralocorticoid Excess Syndrome (AME), which is a congenital syndrome resulting in hypokaleamia and severe hypertension. Patients have elevated cortisol levels in mineralocorticoid target tissues due to reduced 11β-HSD-2 activity. The AME symptoms may also be induced by administration of 11β-HSD-2 inhibitor, glycyrrhetinic acid. The activity of 11β-HSD-2 in placenta is probably important for protecting the fetus from excess exposure to maternal glucocorticoids, which may result in hypertension, glucose intolerance and growth retardation.

Since glucocorticoids are potent regulators of glucose and lipid metabolism, excessive glucocorticoid action may lead to insulin resistance, type 2 diabetes, dyslipidemia, visceral obesity and hypertension. The present invention relates to the use of an 11β-HSD-1 inhibitor for the treatment, control, amelioration, and/or delay of onset of diseases and conditions that are mediated by excessive, or uncontrolled, amounts of cortisol and/or other corticosteroids in a patient by the administration of a therapeutically effective amount of an 11β-HSD-1 inhibitor. Inhibition of the 11β-HSD-1 enzyme limits the conversion of inactive cortisone to active cortisol. Cortisol may cause, or contribute to, the symptoms of these diseases and conditions if it is present in excessive amounts.

The compounds of this invention are 11β-HSD-1 selective inhibitors when comparing to 11β-HSD-2. Previous studies (B. R. Walker et al., J. of Clin. Endocrinology and Met., 80: 3155-3159, 1995) have demonstrated that administration of 11β-HSD-1 inhibitors improves insulin sensitivity in humans. However, these studies were carried out using the nonselective 11β-HSD-1 inhibitor carbenoxolone. Inhibition of 11β-HSD-2 by carbenoxolone causes serious side effects, such as hypertension.

Although cortisol is an important and well-recognized anti-inflammatory agent (J. Baxer, Pharmac. Ther., 2:605-659, 1976), if present in large amount, it also has detrimental effects. For example, cortisol antagonizes the insulin effect in liver resulting in reduced insulin sensitivity and increased gluconeogenesis. Therefore, patients who already have impaired glucose tolerance have a greater probability of developing type 2 diabetes in the presence of abnormally high levels of cortisol.

Glucocorticoids may bind to and activate GRs (and possibly mineralocorticoid receptors) to potentiate the vasoconstrictive effects of both catecholamines and angiotensin II (M. Pirpiris et al., Hypertension, 19:567-574, 1992, C. Kornel et al., Steroids, 58: 580-587, 1993, B. R. Walker and B. C. Williams, Clin. Sci. 82:597-605, 1992). 11β-HSD-1 is present in vascular smooth muscle, which is believed to control the contractile response together with 11β-HSD-2. High levels of cortisol in tissues where the mineralocorticoid receptor is present may lead to hypertension. Therefore, administration of therapeutic dose of an 11β-HSD-1 inhibitor should be effective in treating or prophylactically treating, controlling, and ameliorating the symptoms of NIDDM. Administration of a therapeutically effective amount of an 11β-HSD-1 inhibitor may actually delay, or prevent the onset of type 2 diabetes.

The effects of elevated levels of cortisol are also observed in patients who have Cushing's syndrome (D. N. Orth, N. Engl. J. Med. 332:791-803, 1995, M. Boscaro, et al., Lancet, 357: 783-791, 2001, X. Bertagna, et al, Cushing's Disease. In: Melmed S., Ed. The Pituitary. 2^{nd} ed. Malden, Mass.: Blackwell; 592-612, 2002), which is a metabolic disease characterized by high levels of cortisol in the blood stream. Patients with Cushing's syndrome often develop type 2 diabetes, obesity, metabolic syndrome and dyslipidemia.

Abdominal obesity is closely associated with glucose intolerance (C. T. Montaque et al., Diabetes, 49: 883-888, 2000), hyperinsulinemia, hypertriglyceridemia, and other factors of metabolic syndrome (also known as syndrome X), such as high blood pressure, elevated VLDL, and reduced HDL. Thus, administration of an effective amount of an 11β-HSD-1 inhibitor may be useful in the treatment or control of obesity by controlling excess cortisol, independent of its effectiveness in treating or prophylactically treating NIDDM. Long-term treatment with an 11β-HSD-1 inhibitor may also be useful in delaying the onset of obesity, or perhaps preventing it entirely if the patients use an 11β-HSD-1 inhibitor in combination with controlled diet and exercise.

By reducing insulin resistance and maintaining serum glucose at normal concentrations, compounds of this invention may also have utility in the treatment and prevention of the numerous conditions that often accompany type 2 diabetes and insulin resistance, including the metabolic syndrome, obesity, reactive hypoglycemia, and diabetic dyslipidemia.

The following diseases, disorders and conditions are relates to type 2 diabetes, and some or all of the these may be treated, controlled, in some cases prevented and/or have their onset delayed by treatment with the compounds of this invention: 1) hyperglycemia, 2) low glucose tolerance, 3) insulin resistance, 4) obesity, 5) lipid disorders, 6) dyslipidemia, 7) hyperlipidemia, 8) hypertriglyceridemia, 9) hypercholesterolemia, 10) low HDL levels, 11) high LDL levels, 12) atherosclerosis and its sequelae, 13) vascular restenosis, 14) pancreatitis, 15) abdominal obesity, 16) neurodegenerative disease, 17) retinopathy, 18) nephropathy, 19) neuropathy, 20) metabolic syndrome and other disorders where insulin resistance is a component.

Much evidence in rodents and humans suggests that prolonged elevation of plasma glucocorticoid levels impairs cognitive function, an effect that becomes more profound with aging (A. M. Issa et al., J. Neurosci., 10:3247-3254, 1990, S. J. Lupien et.al., Nat. Neurosci., 1:69-73 1998, J. L. Yau et al., Neuroscience, 66: 571-581, 1995). Chronic excessive cortisol levels in the brain may result in neuronal loss and neuronal dysfunction (D. S. Kerr et al., Psychobiology 22: 123-133, 1994, C. Woolley, Brain Res. 531: 225-231, 1990, P. W. Landfield, Science, 272: 1249-1251, 1996). Therefore, administration of a therapeutic dose of an 11β-HSD-1 inhibitor may result in reduction, amelioration, control and/or prevention of cognitive impairment associated with aging and of neuronal dysfunction.

In Cushing's patients excess cortisol causes hypertension (D. N. Orth, N. Engl. J. Med. 332:791-803, 1995, M. Boscaro, et al., Lancet, 357: 783-791, 2001, X. Bertagna, et al, Cushing's Disease. In: Melmed S., Ed. The Pituitary. 2^{nd} ed. Malden, Mass.: Blackwell; 592-612, 2002). Since hypertension and dyslipidemia contribute to the development of atherosclerosis, administration of a therapeutically effective amount of an 11β-HSD-1 inhibitor of this invention may be beneficial in treating or prophylactically treating, controlling, delaying the onset of, and/or preventing atherosclerosis.

It has been reported that conversion of dehydrocorticosterone to corticosterone by 11β-HSD-1 inhibits insulin secretion from isolated murine pancreatic beta cells (B. Davani et al., J. Biol. Chem., 275: 34841-34844, 2000). Incubation of isolated islets with an 11β-HSD-1 inhibitor improves glucose stimulated insulin secretion. An earlier study suggested that glucocorticoids reduce insulin secretion in vivo (B. Billaudel et al., Horm. Metab. Res. 11: 555-560, 1979). Therefore, inhibition of 11β-HSD-1 enzyme in the pancreas may improve glucose stimulated insulin release.

In clinical ophthalmology, one of the most significant complications caused by using topical and systemic glucocorticoids is corticosteroid-induced glaucoma. This condition is characterized by a significant increase in intraocular pressure (IOP). A recent study indicates that administration of a non-specific 11β-HSD-1 inhibitor, carbenoxolone, to healthy volunteers for seven days resulted in a 17% reduction of IOP. Therefore, administration of 11β-HSD-1 specific inhibitors could be used for the treatment of glaucoma.

In certain disease states, such as tuberculosis, psoriasis, and stress in general, high glucocorticoid activity shifts the immune response to a humoral response, when in fact a cell based response may be more beneficial to the patients. Inhibition of 11β-HSD-1 activity may reduce glucocorticoid levels, thereby shifting the immuno response to a cell based response. (D. Mason, Immunology Today, 12: 57-60, 1991, G. A. W. Rook, Baillier's Clin. Endocrinol. Metab. 13: 576-581, 1999). Therefore, administration of 11β-HSD-1 specific inhibitors could be useful for the treatment of tuberculosis, psoriasis, stress in general, and conditions where a cell based response may be more beneficial than a humoral immune response.

Excess glucocorticoids decrease bone mineral density and increase fracture risk. This effect is mainly mediated by inhibition of osteoblastic bone formation, which results in a net bone loss (C. H. Kim et al. J. Endocrinol. 162: 371-379, 1999,C. G. Bellows et al. 23: 119-125, 1998, M. S. Cooper et al., Bone 27: 375-381, 2000). Therefore, reduction of cortisol levels by administration of an 11β-HSD-1 specific inhibitor may be useful for preventing bone loss due to osteoporosis.

Therapeutic Compositions-Administration-Dose Ranges

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions may be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms may contain diluerts and/or solubilizing or emulsifying agents. Besides inert diluents, the oral compositions may include wetting, emulsifying, sweetening, flavoring, and perfuming agents.

Injectable preparations of the present compounds comprise sterile, injectable, aqueous and oleaginous solutions, suspensions or emulsions, any of which may be optionally formulated with parenterally suitable diluents, dispersing, wetting, or suspending agents. These injectable preparations may be sterilized by filtration through a bacterial-retaining filter or formulated with sterilizing agents that dissolve or disperse in the injectable media.

The absorption of the compounds of the present invention may be delayed by using a liquid suspension of crystalline or amorphous material with poor water solubility. The rate of absorption of the compounds depends upon their rate of dissolution that, in turn, depends on their crystallinity. Delayed absorption of a parenterally administered compound may be accomplished by dissolving or suspending the compound in oil. Injectable depot forms of the compounds may also be prepared by microencapsulating the same in biodegradable polymers. Depending upon the ratio of compound to polymer and the nature of the polymer employed, the rate of release may be controlled. Depot injectable formulations are also prepared by entrapping the compounds in liposomes or microemulsions that are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient may also contain buffering agents. Suppositories for rectal administration may be prepared by mixing the compounds with a suitable non-irritating excipient that is solid at ordinary temperature but fluid in the rectum.

The present compounds may be micro-encapsulated with one or more of the excipients discussed previously. The solid dosage forms of tablets, dragees, capsules, pills, and granules may be prepared with coatings and shells such as enteric and release-controlling. In these forms, the compounds may be mixed with at least one inert diluent and may optionally comprise tableting lubricants and aids. Capsules may also optionally contain opacifying agents that delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers may also be used to increase the flux of the compounds across the skin, and the rate of absorption may be controlled by providing a rate controlling membrane or by dispersing the compounds in a polymer matrix or gel.

Disorders may be treated and/or prophylactically treated in a patient by administering to the patient a therapeutically effective amount of compound of the present invention in such an amount and for such time as is necessary to achieve the desired result. The term “therapeutically effective amount,” refers to administration of a sufficient amount of a compound of formula (I-X) to effectively treat and/or prophylactically treat disorders modulated by the 11-beta-hydroxysteroid dehydrogenase type 1 enzyme at a reasonable benefit/risk ratio applicable to medical treatments. The specific therapeutically effective dose level for any patient population may depend upon one or more factors including, but not limited to, the disorder being treated; the severity of the disorder; the activity of the compound employed; the specific composition employed; age; body weight; general health; gender; diet; time of administration; route of administration; rate of excretion; treatment duration; drugs used in combination; and, coincidental therapy.

The present invention also includes pharmaceutically active metabolites formed by in vivo biotransformation of compounds of formula (I-X). The term “therapeutically suitable metabolite”, as used herein, refers to a pharmaceutically active compound formed by the in vivo biotransformation of compounds of formula (I-X), such as, adamantane hydroxylation and polyhydroxylation metabolites. A discussion of biotransformation is provided in Goodman and Gilman's, The Pharmacological Basis of Therapeutics, seventh edition, MacMillan Publishing Company, New York, N.Y., (1985).

The total daily dose of the compounds of the present invention to effectively inhibit the action of 11-beta-hydroxysteroid dehydrogenase type 1 enzyme in single or divided doses range from about 0.01 mg/kg/day to about 50 mg/kg/day body weight. More preferably, the single or multiple dose ranges from about 0.1 mg/kg/day to about 25 mg/kg/day body weight. Single dose compositions may contain such amounts or multiple doses thereof of the compounds of the present invention to make up the daily dose. In general, treatment regimens comprise administration to a patient from about 10 mg to about 1000 mg of the compounds per day in single or multiple doses.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A method of inhibiting the 11-beta-hydroxysteroid dehydrogenase Type I enzyme in a mammal, comprising administering a therapeutically effective amount of a compound of formula (I), 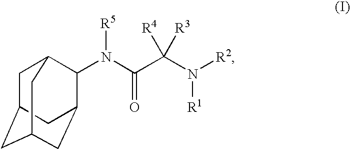
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and any intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and any intervening atoms form a cycloalkyl; R³, R⁴ and any intervening atoms form a non-aromatic heterocycle; and, R², R³ and any intervening carbon and nitrogen atoms form a non-aromatic heterocycle; and,
R⁵ is a member selected from the group consisting of hydrogen, alkyl, carboxyalkyl, carboxycycloalkyl, cycloalkyl, aryl, arylalkyl, aryloxyalkyl, heterocycle, heterocyclealkyl, and, heterocycleoxyalkyl.

2. The method according to claim 1, comprising administering a therapeutically effective amount of a prodrug of the compound of formula (I).

3. The method according to claim 1, comprising administering a therapeutically effective amount of a salt of the compound of formula (I).

4. A method of inhibiting the 11-beta-hydroxysteroid dehydrogenase Type I enzyme in a mammal, comprising administering a therapeutically effective amount of a compound of formula (II), 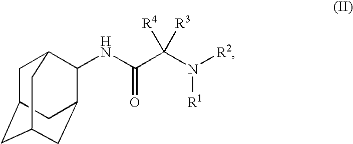
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle; and, R¹, R² and any intervening atoms form a heterocycle; and,
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and any intervening atoms form a cycloalkyl; R³, R⁴ and any intervening atoms form anon-aromatic heterocycle; and, R², R³ and any intervening carbon and nitrogen atoms form a non-aromatic heterocycle.

5. The method according to claim 4, comprising administering a therapeutically effective amount of a prodrug of the compound of formula (II).

6. The method according to claim 4, comprising administering a therapeutically effective amount of a salt of the compound of formula (II).

7. A method of inhibiting the 11-beta-hydroxysteroid dehydrogenase Type I enzyme in a mammal, comprising administering a therapeutically effective amount of a compound of formula (III), 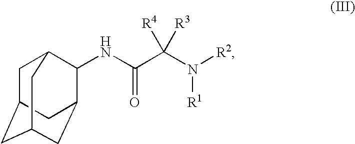
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle; and,
R³ and R⁴ are each a member independently selected from the group consisting of hydrogen, alkyl, carboxyalkyl, carboxycycloalkyl, cycloalkyl, aryl, and, heterocycle.

8. The method according to claim 7, comprising administering a therapeutically effective amount of a prodrug of the compound of formula (III).

9. The method according to claim 7, comprising administering a therapeutically effective amount of a salt of the compound of formula (III).

10. A method of inhibiting the 11-beta-hydroxysteroid dehydrogenase Type I enzyme in a mammal, comprising administering a therapeutically effective amount of a compound of formula (IV), 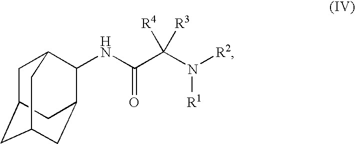
wherein
R¹, R² and any intervening atoms form a heterocycle; and,
R³ and R⁴ are each a member independently selected from the group consisting of hydrogen, alkyl, carboxyalkyl, carboxycycloalkyl, cycloalkyl, aryl, and, heterocycle.

11. The method according to claim 10, wherein the compound is
N-2-adamantyl-2-{4-[2-(benzyloxy)ethyl]piperazin-1-yl}acetamide;
N-2-adamantyl-2-[4-(2-furoyl)piperazin-1-yl]propanamide;
N-2-adamantyl-2-(4-hydroxypiperidin-1-yl)propanamide; or
N-2-adamantyl-2-methyl-2-piperidin-1-ylpropanamide.

12. The method according to claim 10, comprising administering a therapeutically effective amount of a prodrug of the compound of formula (IV).

13. The method according to claim 10, comprising administering a therapeutically effective amount of a salt of the compound of formula (IV).

14. A method of inhibiting the 11-beta-hydroxysteroid dehydrogenase Type I enzyme in a mammal, comprising administering a therapeutically effective amount of a compound of formula (V), 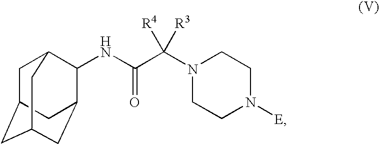
wherein
R³ and R⁴ are each a member independently selected from the group consisting of hydrogen, alkyl, carboxyalkyl, carboxycycloalkyl, cycloalkyl, aryl, heterocycle; R³, R⁴and any intervening atoms form a cycloalkyl; and, R³, R⁴ and any intervening atoms form a non-aromatic heterocycle; and,
E is a member selected from the group consisting of aryl and heterocycle.

15. The method according to claim 14, wherein the compound is
N-2-adamantyl-2-[4-(5-chloropyridin-2-yl)piperazin-1-yl]acetamide;
N-2-adamantyl-2-[4-(5-chloropyridin-2-yl)piperazin-1-yl]propanamide; or
N-2-adamantyl-2-methyl-2-{4-[5-(trifluoromethyl)pyridin-2-yl]piperazin-1-yl}propanamide.

16. The method according to claim 14, comprising administering a therapeutically effective amount of a prodrug of the compound of formula (V).

17. The method according to claim 14, comprising administering a therapeutically effective amount of a salt of the compound of formula (V).

18. A method of inhibiting the 11-beta-hydroxysteroid dehydrogenase Type I enzyme in a mammal, comprising administering a therapeutically effective amount of a compound of formula (VI), 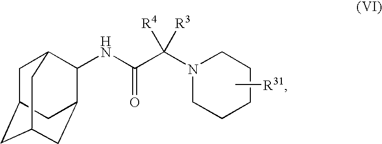
wherein
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and any intervening atoms form a cycloalkyl; R³, R⁴ and any intervening atoms form anon-aromatic heterocycle; and, R², R³ and any intervening carbon and nitrogen atoms form a non-aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

19. The method according to claim 18, comprising administering a therapeutically effective amount of a prodrug of the compound of formula (VI).

20. The method according to claim 18, comprising administering a therapeutically effective amount of a salt of the compound of formula (VI).

21. A method of inhibiting the 11-beta-hydroxysteroid dehydrogenase Type I enzyme in a mammal, comprising administering a therapeutically effective amount of a compound of formula (VII), 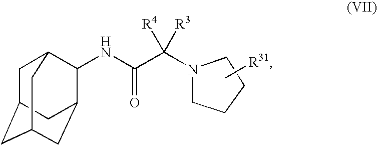
wherein
R³ and R⁴ are each a member independently selected from the group consisting of hydrogen, alkyl, carboxyalkyl, carboxycycloalkyl, cycloalkyl, haloalkyl, aryl, heterocycle; R³, R⁴ and any intervening atoms form a cycloalkyl; and, R³, R⁴ and any intervening atoms form a non-aromatic heterocycle; and,
R³¹ is a member selected from the group consisting of alkyl, alkoxy, aryl, arylalkyl, aryloxy, aryloxyalkyl, halogen, haloalkyl, heterocycle, heterocyclealkyl, heterocycleoxy, heterocycleoxyalkyl, and, hydroxy.

22. The method according to claim 21, comprising administering a therapeutically effective amount of a prodrug of the compound of formula (VII).

23. The method according to claim 21, comprising administering a therapeutically effective amount of a salt of the compound of formula (VII).

24. A method of inhibiting the 11-beta-hydroxysteroid dehydrogenase Type I enzyme in a mammal, comprising administering a therapeutically effective amount of a compound of formula (VIII), 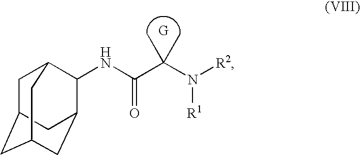
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle; and,
G is selected from the group consisting of a cycloalkyl and a non-aromatic heterocycle.

25. The method according to claim 24, comprising administering a therapeutically effective amount of a prodrug of the compound of formula (VIII).

26. The method according to claim 24, comprising administering a therapeutically effective amount of a salt of the compound of formula (VIII).

27. A method of inhibiting the 11-beta-hydroxysteroid dehydrogenase Type I enzyme in a mammal, comprising administering a therapeutically effective amount of a compound of formula (IX), 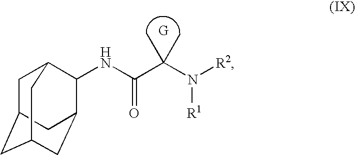
wherein
R¹, R² and any intervening atoms form a heterocycle; and,
G is selected from the group consisting of a cycloalkyl and a non-aromatic heterocycle.

28. The method according to claim 27, comprising administering a therapeutically effective amount of a prodrug of the compound of formula (IX).

29. The method according to claim 27, comprising administering a therapeutically effective amount of a salt of the compound of formula (IX).

30. A method of inhibiting the 11-beta-hydroxysteroid dehydrogenase Type I enzyme in a mammal, comprising administering a therapeutically effective amount of a compound of formula (X), 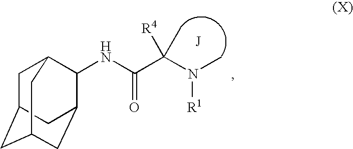
wherein
R¹ is a member selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle;
R⁴ is a member selected from the group consisting of hydrogen, alkyl, carboxyalkyl, carboxycycloalkyl, cycloalkyl, aryl, and, heterocycle; and,
J is a non-aromatic heterocycle.

31. The method according to claim 30, wherein the compound of formula (X) is a member selected from the group consisting of:
N-2-adamantyl-1-(pyridin-2-ylmethyl)piperidine-2-carboxamide;
4-({2-[(2-adamantylamino)carbonyl]pyrrolidin-1-yl}methyl)benzoic acid; and
N-2-adamantyl-1-[4-(aminocarbonyl)benzyl]prolinamide.

32. The method according to claim 30, comprising administering a therapeutically effective amount of a prodrug of the compound of formula (X).

33. The method according to claim 30, comprising administering a therapeutically effective amount of a salt of the compound of formula (X).

34. The method according to claim 1, further comprising the step of treating or prophylactically treating non-insulin dependent type 2 diabetes.

35. The method according to claim 1, further comprising the step of treating or prophylactically treating insulin resistance.

36. The method according to claim 1, further comprising the step of treating or prophylactically treating obesity

37. The method according to claim 1, further comprising the step of treating or prophylactically treating lipid disorders.

38. The method according to claim 1, further comprising the step of treating or prophylactically treating metabolic syndrome.

39. The method according to claim 1, further comprising the step of treating or prophylactically treating any disease or condition mediated by excessive glucocorticoid action.

40. The method according to claim 4, further comprising the step of treating or prophylactically treating non-insulin dependent type 2 diabetes.

41. The method according to claim 4, further comprising the step of treating or prophylactically treating insulin resistance.

42. The method according to claim 4, further comprising the step of treating or prophylactically treating obesity

43. The method according to claim 4, further comprising the step of treating or prophylactically treating lipid disorders.

44. The method according to claim 4, further comprising the step of treating or prophylactically treating metabolic syndrome.

45. The method according to claim 4, further comprising the step of treating or prophylactically treating any disease or condition mediated by excessive glucocorticoid action.

46. The method according to claim 7, further comprising the step of treating or prophylactically treating non-insulin dependent type 2 diabetes.

47. The method according to claim 7, further comprising the step of treating or prophylactically treating insulin resistance.

48. The method according to claim 7, further comprising the step of treating or prophylactically treating obesity

49. The method according to claim 7, further comprising the step of treating or prophylactically treating lipid disorders.

50. The method according to claim 7, further comprising the step of treating or prophylactically treating metabolic syndrome.

51. The method according to claim 7, further comprising the step of treating or prophylactically treating any disease or condition mediated by excessive glucocorticoid action.

52. The method according to claim 10, further comprising the step of treating or prophylactically treating non-insulin dependent type 2 diabetes.

53. The method according to claim 10, further comprising the step of treating or prophylactically treating insulin resistance.

54. The method according to claim 10, further comprising the step of treating or prophylactically treating obesity

55. The method according to claim 10, further comprising the step of treating or prophylactically treating lipid disorders.

56. The method according to claim 10, further comprising the step of treating or prophylactically treating metabolic syndrome.

57. The method according to claim 10, further comprising the step of treating or prophylactically treating any disease or condition mediated by excessive glucocorticoid action.

58. The method according to claim 14, further comprising the step of treating or prophylactically treating non-insulin dependent type 2 diabetes.

59. The method according to claim 14, further comprising the step of treating or prophylactically treating insulin resistance.

60. The method according to claim 14, further comprising the step of treating or prophylactically treating obesity

61. The method according to claim 14, further comprising the step of treating or prophylactically treating lipid disorders.

62. The method according to claim 14, further comprising the step of treating or prophylactically treating metabolic syndrome.

63. The method according to claim 14, further comprising the step of treating or prophylactically treating any disease or condition mediated by excessive glucocorticoid action.

64. The method according to claim 18, further comprising the step of treating or prophylactically treating non-insulin dependent type 2 diabetes.

65. The method according to claim 18, further comprising the step of treating or prophylactically treating insulin resistance.

66. The method according to claim 18, further comprising the step of treating or prophylactically treating obesity

67. The method according to claim 18, further comprising the step of treating or prophylactically treating lipid disorders.

68. The method according to claim 18, further comprising the step of treating or prophylactically treating metabolic syndrome.

69. The method according to claim 18, further comprising the step of treating or prophylactically treating any disease or condition mediated by excessive glucocorticoid action.

70. The method according to claim 21, further comprising the step of treating or prophylactically treating non-insulin dependent type 2 diabetes.

71. The method according to claim 21, further comprising the step of treating or prophylactically treating insulin resistance.

72. The method according to claim 21, further comprising the step of treating or prophylactically treating obesity

73. The method according to claim 21, further comprising the step of treating or prophylactically treating lipid disorders.

74. The method according to claim 21, further comprising the step of treating or prophylactically treating metabolic syndrome.

75. The method according to claim 21, further comprising the step of treating or prophylactically treating any diseases or condition mediated by excessive glucocorticoid action.

76. The method according to claim 24, further comprising the step of treating or prophylactically treating non-insulin dependent type 2 diabetes.

77. The method according to claim 24, further comprising treat, prophylactically treat or prevent insulin resistance.

78. The method according to claim 24, further comprising the step of treating or prophylactically treating obesity

79. The method according to claim 24, further comprising the step of treating or prophylactically treating lipid disorders.

80. The method according to claim 24, further comprising the step of treating or prophylactically treating metabolic syndrome.

81. The method according to claim 24, further comprising the step of treating or prophylactically treating any disease or condition mediated by excessive glucocorticoid action.

82. The method according to claim 27, wherein the inhibition of the 11-beta-hydroxysteroid dehydrogenase Type 1 enzyme in a mammal treats, prophylactically treats or prevents non-insulin dependent type 2 diabetes.

83. The method according to claim 27, further comprising the step of treating or prophylactically treating insulin resistance.

84. The method according to claim 27, further comprising the step of treating or prophylactically treating obesity

85. The method according to claim 27, further comprising the step of treating or prophylactically treating lipid disorders.

86. The method according to claim 27, further comprising the step of treating or prophylactically treating metabolic syndrome.

87. The method according to claim 27, further comprising the step of treating or prophylactically treating a disease or condition mediated by excessive glucocorticoid action.

88. The method according to claim 30, further comprising the step of treating or prophylactically treating non-insulin dependent type 2 diabetes.

89. The method according to claim 30, further comprising the step of treating or prophylactically treating insulin resistance.

90. The method according to claim 30, further comprising the step of treating or prophylactically treating obesity.

91. The method according to claim 30, further comprising the step of treating or prophylactically treating lipid disorders.

92. The method according to claim 30, further comprising the step of treating or prophylactically treating metabolic syndrome.

93. The method according to claim 30, further comprising the step of treating or prophylactically treating a disease or condition mediated by excessive glucocorticoid action.
